(19) United States
(12) Patent Application Publication
(10) Pub. No.: US 2015/0268184 A1
(43) Pub. Date: Sep. 24, 2015

(54) GAS SENSORS USING MAGNETIC FIELDS AND METHODS OF USE THEREOF

(71) Applicant: GEORGIA TECH RESEARCH CORPORATION, Atlanta, GA (US)

(72) Inventors: James Gole, Atlanta, GA (US); Caitlin V. Baker, Atlanta, GA (US); William Ivey Laminack, Atlanta, GA (US); Travis C. Tune, Murfreesboro, TN (US)

(21) Appl. No.: 14/336,022

(22) Filed: Jul. 21, 2014

Related U.S. Application Data
(60) Provisional application No. 61/954,633, filed on Mar. 18, 2014.

(51) Int. Cl. G01N 27/02; G01N 33/00; G01N 27/72

(52) U.S. Cl. CPC G01N 27/02; G01N 27/72; G01N 33/0027

(57) ABSTRACT

Embodiments of the present disclosure include sensors, arrays of sensors, devices including sensors, methods of making sensors, methods of using sensors, and the like, whereupon exposure to a magnetic field results in embodiments having enhanced sensitivity. In an embodiment, the present disclosure includes porous silicon (PS) sensors, arrays of PS sensors, devices including PS sensors, methods of making PS sensors, methods of using PS gas sensors, and the like, that include or use a magnetic field.

### CROSS-REFERENCE TO RELATED APPLICATION

In addition, this application claims priority to co-pending U.S. Provisional Application entitled “Magnetically Induced Enhancement of Reversibly Responding Coductormetric Sensors” having Ser. No. 61/954,633, filed on Mar. 18, 2014, which is incorporated herein by reference.

### BACKGROUND

Porous silicon (PS) has drawn considerable attention for sensor applications. Its luminescence properties, large surface area, and compatibility with silicon based technologies have been the driving force for this technology development. However, there exists a need in the industry to advance sensor technologies.

### SUMMARY

Embodiments of the present disclosure include sensors, arrays of sensors, devices including sensors, methods of making sensors, methods of using sensors, and the like, whereupon exposure to a magnetic field results in embodiments having enhanced sensitivity. In an embodiment, the present disclosure includes porous silicon (PS) sensors, arrays of PS sensors, devices including PS sensors, methods of making PS sensors, methods of using PS gas sensors, and the like, that include or use a magnetic field.

One exemplary embodiment of a device, among others, includes: a magnetic system including a magnet, and a conductometric porous silicon gas sensor positioned relative to the magnet so that the sensor is exposed to a magnetic field of the magnet, wherein the conductometric porous silicon gas sensor includes a silicon substrate having a porous silicon layer, wherein a plurality of magnetic nanostructures are disposed on a portion of the porous silicon layer to provide a fractional coverage on the porous silicon layer, wherein the conductometric porous silicon gas sensor is operative to transduce the presence of a gas into an impedance change when exposed the magnetic field, wherein the impedance change correlates to the gas concentration. In an embodiment, the magnetic nanostructure is a paramagnetic, ferrimagnetic, or ferromagnetic nanostructure. In an embodiment, the magnetic nanostructure has a paramagnetic property, ferromagnetic property, or ferromagnetic property.

One exemplary embodiment of a method of detecting a concentration of a gas, among others, includes: providing a magnetic system including a magnet and a conductometric porous silicon gas sensor positioned relative to the magnet so that the sensor is exposed to a magnetic field of the magnet, wherein the conductometric porous silicon gas sensor including a silicon substrate having a porous silicon layer, wherein a plurality of magnetic nanostructures are disposed on a portion of the porous silicon layer to provide a fractional coverage on the porous silicon layer, wherein the conductometric porous silicon gas sensor is operative to transduce the presence of a gas into an impedance change when exposed to the magnetic field, wherein the impedance change correlates to the gas concentration: exposing the porous silicon layer to the magnetic field so that the porous silicon layer is about 200 to 1000 Gauss; introducing the gas to the sensor; and measuring an impedance change in the sensor. In an embodiment, the magnetic nanostructure is a paramagnetic, ferrimagnetic, or ferromagnetic nanostructure. In an embodiment, the magnetic nanostructure has a paramagnetic property, ferromagnetic property, or ferromagnetic property.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1(A) illustrates a classical iron magnet configuration. The iron magnet can be rotated in the direction of the sensor, which is monitored with two precision microprobes as a gas flow of entrained NO intersects the sensor. FIG. 1(B) Neodymium rare earth magnet configuration interacting from below and corresponding to ˜2000 Gauss. This configuration is distinct from that in FIG. 1(A). The configuration of the magnets is relevant to the interaction observed for these systems.

FIG. 2 illustrates the magnetic field effect on a Co(II)Cl₂.6 H₂O treated n-type PS interface. (a) response of PS sensor interface to NO (red) indicating that NO is extracting electrons (resistance increases) and acting as an acid, (b) response after treatment with Co(II) (green), and (c) response after introduction of an 80 Gauss magnetic field (blue).

FIG. 3 illustrates the magnetic field effect on a Co(II)Cl₂.(6−x(x>2)) H₂O treated n-type PS interface. (a) response of PS sensor interface to NO (red) indicating that NO is extracting electrons (resistance increases) and acting as an acid, (b) response after treatment with Co(II) (green), and (c) response after introduction of an 80 Gauss magnetic field.

FIG. 4 illustrates the response of a p-type PS sensor interface to NO (a) without the presence of a magnetic field (blue) and (b) after introduction of a 435 Gauss magnetic field (green).

FIG. 5 illustrates the magnetic field effect on a TiO₂ treated n-type PS interface. (a) response of TiO₂ PS sensor interface to NO (green) indicating that NO is extracting electrons (resistance increases) and acting as an acid, and (b) response after introduction of an 80 Gauss magnetic field.

FIG. 6 illustrates the magnetic field effect on a Co(II)Cl₂.(6) H₂O treated p-type PS interface. (a) response of PS sensor interface to NO (blue) indicating that NO is extracting electrons (resistance decreases) and acting as an acid, (b) response after treatment with Co(II) (green) at a concentration leading to a decrease in sensor response at a concentration sufficiently high so as to lead to cross talk among nanostructures, and (c) response after introduction of an 80 Gauss magnetic field. The enhancement with magnetic field is evident.

FIG. 7 illustrates the magnetic field effect on a Co(II)Cl₂.(6) H₂O treated p-type PS interface. (a) response of PS sensor interface to NO (blue) indicating that NO is extracting electrons (resistance decreases) and acting as an acid, (b) response after treatment with Co(II) (green) at a concentration approximately half that of FIG. 6 and showing a significant sensor enhancement, and (c) response after introduction of a 200 Gauss magnetic field. The lack of an enhancement with magnetic field is evident.

FIG. 8 illustrates the magnetic field effect on an iron oxide nanostructure treated (0.05-see exptl.) p-type PS interface. (a) response of PS sensor interface to NO (blue) indicating that NO is extracting electrons (resistance decreases) and acting as an acid, (b) response after treatment with Fe(II) (green) at a concentration leading to a decrease in sensor response at a concentration sufficiently high so as to lead to cross talk among nanostructures, and (c) response after introduction of an ˜2000 Gauss magnetic field (red). There is an enhancement with magnetic field relative to the interactive iron oxide deposition, however, the response is considerably less than that of the PS interface.

FIG. 9 illustrates the magnetic field effect on an iron oxide nanostructure treated (0.045-see exptl.) p-type PS interface. (a) response of PS sensor interface to NO (blue) indicating that NO is extracting electrons (resistance decreases) and acting as an acid, (b) response after treatment with Fe(II) (green) at a concentration leading to an increase in sensor response at a deposition sufficiently low so as to avoid cross talk among nanostructures, and (c) response after introduction of an ˜2000 Gauss magnetic field (red). See text for discussion.

FIG. 10 illustrates the magnetic field effect on an iron oxide nanostructure treated (0.045-see exptl.) p-type PS interface. (a) response of PS sensor interface to NO (blue) indicating that NO is extracting electrons (resistance decreases) and acting as an acid, (b) response after treatment with Fe(II) (green) at a concentration leading to an increase in sensor response at a concentration sufficiently low so as to avoid cross talk among nanostructures, and (c) response after introduction of an ˜1000 Gauss magnetic field (red). The response is distinct from the 2000 Gauss magnet of FIG. 9.

FIG. 11 illustrates the Effect of an iron oxide nanostructure treated (0.040-see exptl.) n-type PS interface. (a) response of PS sensor interface to NO (blue) indicating that NO is extracting electrons (resistance decreases) and acting as an acid, (b) response after treatment with Fe(II) (green) at a concentration leading to an increase in sensor response at a concentration sufficiently low so as to avoid cross talk among nanostructures. The response increases significantly.

FIG. 12 illustrates the magnetic field effect on an iron oxide nanostructure treated (0.040—see exptl.) n-type PS interface. (a) response of PS sensor interface to NO (blue) indicating that NO is extracting electrons (resistance decreases) and acting as an acid, (b) response after treatment with Fe(II) (green) at a concentration leading to an increase in sensor response at a concentration sufficiently low so as to avoid cross talk among nanostructures, and (c) response after introduction of an ˜2000 Gauss magnetic field (red). The response increases significantly with magnetic field.

FIG. 13 is a schematic of the Fermi-Dirac probability function at T=0° K and T>0° K.

FIG. 14 illustrates the (A) schematic of lattice for an n-type semiconductor (a free electron is donated by phosphorous); (B) donor levels at absolute zero and (c) above absolute zero²⁰.

FIG. 15 illustrates the estimated hard and soft acidities and basicities based on resistance changes relative to a p- and n-type porous silicon interface¹¹.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that any pair of intervening values in the stated range are encompassed within the disclosure although they may not be specifically recited, subject to any specifically excluded limit in the stated range (e.g., if the range is 1 to 100, then the range of 10 to 20 is also included and can be claimed even if not specifically recited).

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material sceince, and the like, which are within the skill of the art. Such techniques are explained fully in the literature.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### Definitions

The term “detectable” refers to the ability to detect a signal over the background signal.

The term “detectable signal” is a signal derived from an impedance change upon the interaction of a gas with a porous silicon layer or a porous silicon layer having a nanostructured deposit on the porous silicon layer. The detectable signal is detectable and distinguishable from other background signals. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, 40% or more difference between the detectable signal and the background) between the detectable signal and the background. Standards and/or calibration curves and/or arrays of porous silicon sensors can be used to determine the relative intensity of the detectable signal and/or the background.

### Discussion

Embodiments of the present disclosure include sensors, arrays of sensors, devices including sensors, methods of making sensors, methods of using sensors, and the like, whereupon exposure to a magnetic field results in embodiments having enhanced sensitivity. In an embodiment, the present disclosure includes porous silicon (PS) sensors, arrays of PS sensors, devices including PS sensors, methods of making PS sensors, methods of using PS gas sensors, and the like, that include or use a magnetic field.

Typically, a sensor is not exposed to a magnetic field due to the nature of its construction and/or how it is typically used. An advantage of an embodiment of the present disclosure is that the sensitity of the sensor can be enhanced (e.g., about 25% or more) when exposed to a magnetic field as compared to the same sensor that is not exposed to the magnetic field. Thus, the sensor, device, or sensor system can be configured so that a region (e.g., porous silicon layer) of the sensor is subject to a selected strength of a magnetic field, for example from a magnet of magnetic system. Additioanl details will be described herein and in the Example.

Embodiments of the present disclosure can be understood based on the Inverse Hard/Soft Acid/Base (IHSAB) principle/approach. The Inverse Hard/Soft Acid/Base (IHSAB) principle/approach, as it correlates with a basis in physisorption (electron transduction), complements the HSAB principle for hard/soft acid/base interactions and can be correlated with a basis in density functional theory (DFT). The basis for correlation follows the principle that soft-soft acid/base interactions produce significant covalent bonding and hard-hard combinations produce significant ionic bonding. The HSAB principle states that hard acids prefer to coordinate to hard bases and soft acids prefer to coordinate to soft bases. In contrast, the driving principle to promote physisorption (electron transduction) represents the inverse of that necessary to form strong chemical bonds. This principle is manifest through the interactions of metal oxide nanostructures as they influence the nature of the majority carriers in a p- or n-type semiconductor. In an embodiment, nanoporous silicon layers positioned on porous silicon (PS) micropores facilitate the deposition of nanostructured metal/metal oxides which provide distinctly higher variable sensitivities and selectivity for a given extrinsic semiconductor interface as well as magnetic characteristic for paramagnetic, ferrimagnetic, and ferromagnetic elements and their ions. In an embodiment, the PS surface is treated with fractional depositions (e.g., islands of nanostructures), much less than a monolayer to insure that the nanostructures do not begin to interact via cross talk and, in so doing diminish signal to noise. This deposition can be made to produce a dominant physisorptive (sensors) or chemisorptive (micro-reactors) character at the semiconductor interface as the deposited nanostructures act to focus the nature of the surface interaction.

However, the nature of the extrinsic semiconductor, the manner in which its donor and/or acceptor levels can be manipulated, and its transformation to intrinsic character also represent important variables. For example, magnetic metal or oxide nanostructures (e.g., paramagnetic, ferrimagnetic, or ferromagnetic) disposed on the porous silicon layer can be subjected to a magnetic field at room temperature to enhance electron transfer that can be used to improve a sensing process by modifying the conductivity due to the concentration of available electrons in an n-type extrinsic semiconductor interface or a p-type extrinsic semiconductor interface. Embodiments of the present disclosure can use this characteristic to enhance (e.g., about 25% or more, about 50% or more, about 100% or more, or about 150% or more) the sensitivity of the sensor relative to the same sensor that is not subject to a magnetic field.

An embodiment of the present disclosure describes a conductometric gas sensor that is operative to measure an impedance change that corresponds to a gas concentration (e.g., a gas concentration can be determined based on the impedance change or the magnitude of the impedance change and this concentration can be independently evaluated for calibration) when subject to a magnetic field. More particularly, the conductometric sensor transduces the presence of a gas into an impedance signal, which is measured by another device in communication with the conductometric sensor. Therefore, the term “measure” used in reference to the conductometric sensor can include the conductometric sensor in combination with another circuit or device (e.g., impedance analyzer, sensor and shunt circuit, and the like) to measure an impedance or resistance change (e.g., the detectable signal). The conductometric sensor can be used to detect gases or liquids. In particular, conductometric sensors, in accordance with the present disclosure, have a rapid and reversible response to analyte gases at room temperature while being subject to a magnetic field.

In an embodiment, the n-type semiconductor or the p-type semiconductor is PS, so the sensor can be referred to as a conductometric PS gas sensor (also referred to as a “PS gas sensor” or “conductometric PS sensor”). Although some embodiments of the present disclosure are described as PS, other n-type semiconductor materials or p-type semiconductor materials can be used that include a nanopore coated micro-porous layer. There is no intention to limit embodiments of the present disclosure to PS materials and other materials, such as those described herein, can be used.

In an embodiment, the conductometric PS gas sensor can be made from an n-type PS substrate or a p-type PS substrate. In an embodiment, a conductometric PS gas sensor made from n-type PS can operate at a lower concentrations than a p-type PS based conductometric PS gas sensors to extend down to the parts per billion (ppb) less than 100 ppb, less than about 75 ppb, less than about 50 ppb), while p-type PS based conductometric PS gas sensors generally operate in a linear fashion at pressures higher (greater than 0.5 ppm) than do n-type PS based conductometric PS gas sensors. Thus, an embodiment of the present disclosure can include both p-type and n-type PS based conductometric PS gas sensors that can be used in a wide dynamic range of gas concentrations. Some detection abilities for some specific gases are given in the Example, and these illustrate the general sensitity of the sensor subject to a magnetic field which can be extended to other gases or gas mixtures.

Embodiments of the present disclosure provide for a concept that is predictive of significant and predictable changes in the conductometric sensor (e.g., conductometric PS sensor) sensitivity for a variety of gases, when decorated (nanostructure islands), and the PS layer is subject to a magnetic field. Rapidly responding, reversible, sensitive, and selective conductometric PS sensors used at room temperature can be formed (1) with a highly efficient electrical contact to a porous silicon layer (e.g., a nanopore covered microporous layer), while also including (2) deposited nanostructures (e.g., nanostructures as nanostructured fractional deposits, which enhance the sensitivity of the sensor subject to a magnetic field), using embodiments of the present disclosure.

In an embodiment, the nanostructures can be deposited as distinctly variable nanostructures that can be chosen to be deposited on a portion (e.g, distinct islands) of the porous silicon layer, where the resulting conductometric PS sensor provides a range of sensitivities for a given analyte using a concept complementary to that of hard and soft acid-base interactions (HSAB) and commensurate with a basis in dominant physisorption. The physisorption interaction involves electron transduction between the gas and nanostructured deposit. The physisorption interaction may involve a change in the electronic orbital patterns of the nanostructured deposit but the key is the analyte gas and orbital miss-match which leads to a weak interation. A physisorption interaction is a reversible interaction of the nanostructured deposit with the gas. A physisorption interaction is not a chemisorption reaction that involves a chemical reaction that likely is not reversible. The concept, based on the reversible interaction of hard acids and bases with soft bases and acids corresponds (1) to the inverse of the HSAB concept and (2) to the selection of a conductometric PS sensor and a porous silicon surface (e.g., nanostructures islands) and analyte materials, which do not undergo strong covalent or ionic bonding but rather represent a much weaker orbital interaction where a significant HOMO-LUMO and additional orbital mismatches dominate the interaction as a reversible physisorption interaction (electon transduction). For example, at 300° K., the donor level population has been depleted sufficiently so that there are a significant number of levels available for population when a basic analyte interacts with the decorated semiconductor interface, contributes electrons to the donor levels, enhances the majority charge carrier concentration, and increases conductance. This process can eventually “top out” the level population. Similarly, an acidic analyte, as it withdraws electrons, decreases conductance, depletes and can eventually “bottom out” the donor level population. Embodiments of the present disclosure provide for notably higher sensitivities and selectivity based on further modifications of the charge carrier populations and impedance changes when subject to a magnetic field.

An embodiment of the present disclosure can be advantageous for one or more of the following reasons: (1) enhanced sensitity when the porous silicon layer is subject to a magnetic field relative to the same sensor that is not subject to the magnetic field, (2) its operation at room temperature as well as at a single, readily accessible, temperature with an insensitivity to temperature drift, (3) its potential operation in a heat-sunk configuration allowing operation to a surface temperature of 80° C. even in highly elevated temperature environments (in sharp contrast to metal oxide sensors), (4) its ease of deposition with a diversity of gas-selective materials to form sensor arrays, (5) its low cost of fabrication, (6) its low cost and ease of rejuvenation after contamination, (7) its low cost of operation, and/or (8) its ability to rapidly assess false positives by operating the sensor in a pulsed gas mode.

It should be noted that the time frame for measuring the gas concentration depends, in part, on the experimental configuration for gas delivery and the particular application and gas being measured. The presence of the gas can be measured in a time frame less than or equal to 2 seconds in most embodiments dictated by the gas delivery system, while in other embodiments, the time frame for a precise concentration measurement may be longer. In an embodiment, the sensor itself responds in less than 2 seconds. It should be noted that impedance includes contributions from one or more of resistance, capacitance, and inductance, and measurement of impedance includes the measurement of one or more of resistance, capacitance, and inductance. In an embodiment, the impedance analyzer measures the resistance and capacitance only.

Embodiments of the present disclosure can be used to measure the concentration of a gas or a mixture of gases when the gas or the gas mixture is known or substantially known, while the porous silicon layer is subject to the magnetic field. For example, if the environment that the conductometric PS sensor is to be used in is known to include ammonia, the conductometric PS sensor can be used to measure the concentration of ammonia. In another example, if the environment that the conductometric PS sensor is to be used is known to include ammonia and H₂S, then an array matrix of conductometric PS sensors (e.g., 2, 3, 4, 6, 8, or more) can be used to measure the concentration of each gas or the relative concentration of each gas, where one or more portions of the array are subject to the same magnetic field or to different magnetic fields. In an embodiment, the array of conductometric PS sensors can be designed so that each of the conductometric PS sensors has a greater sensitivity for detection of one specific gas over another. In addition, the array of conductometric PS sensors allows the concentration of the gases to be compared to one another on relative terms.

In an embodiment, the conductometric PS sensor includes a silicon substrate (n-type or p-type), a protective layer on a portion of the silicon substrate, an n-type PS layer (or region) or p-type PS layer (or region)) on a portion of the silicon substrate that is not covered by the protective layer, and two or more distinct gold contacts disposed onto a portion of the PS region and the protective layer. A plurality of nanostructures (sometimes referred to as a “nanostructured deposits”) can be disposed in a fractional manner (e.g., non-contiguous islands) on and/or within the PS layer that is not covered by the contacts, which enables the conductometric PS sensor to respond more strongly to certain gases relative to others depending on the nanostructures used. In some instances, reference is made to “porous silicon layer”, “PS layer”, “n-type PS layer”, or “p-type PS layer”, and unless contrary to the specific discussion or description (e.g., specific discussions in the Example, for example how each type of layer operates when subjected to a magnetic field, use of a particular type of magnetic nanoparticle (e.g., paramagnetic, ferrimagnetic, or ferromagnetic nanoparticle), a particular gas, and the like), such reference refers to “n-type PS layer”, or “p-type PS layer” and is not restricted to one or the other so long as the sensor operates as it is intended to operate.

In an embodiment, the device and/or system including the conductometric PS sensor is configured so that the PS layer can be subject to the magnetic field. In an embodiment, the magnet field can have an energy of about 100 to 2000 Gauss or about 100 to 1000 Gauss. In an embodiment, the magnetic field can be adjusted according to the desired detection level, so in some instances a low magnetic field energy (e.g., 1000 Gauss or less) is sufficient to achieve the desired detection level.

In an embodiment, the device and/or system includes a controllable magnetic system(s) that includes a magnet(s) positioned so that the porous silicon layer can be subjected to the magnetic field at a particular strength. In an embodiment, the device, system, and/or sensor can be configured to allow for the PS layer to be subjected to the magnetic field from the surrounding area. For example, the device, system, and/or sensor can be configured so that the magnetic system is physically part of the device, system, or sensor or can be physically separated, and in either configuration, the porous silicon layer can be subjected to the magnetic field.

In an embodiment, the magnetic system can include a magnet and appropriate control system (e.g, computer) to operate the magnet. In an embodiment, the magnetic system can be interfaced with a system that receives the data from the sensor, so that varibles can be adjusted as needed to operate the sensor. For example, the magnetic field can be adjusted based on the information received from the sensor and in a particular embodiment, the magnetic field can be adjusted to scan across a certain magnetic field strength range.

In an embodiment, the magnet can be a permanent magnet, electromagnet, or the like. In an embodiment, the magnet can be a ferrite magnet, rare earth magnet, a neodymium rare earth magnet, samarium cobalt magnet, alnico magnet, ceramic magnet, and the like. The magnet can be positioned relative (e.g., in front, back, or the side, a few mm, to cm, to a meter or more away) to the sensor to apply a magnetic field of appropriate strength onto the decorated porous silicon layer to achieve enhanced sensitity in the present disclosure. The exact position of the magnet is less important than the magnetic field applied to the sensor, and both can be adjusted as desired to achieve the desired characteristics of the sensor.

In an embodiment, the protective layer can include, but is not limited to, a silicon carbide layer, a silicon nitride layer, a silicon oxynitride (SiOₓN_{y}) layer, an insulating dielectric film, a ceramic layer, and combinations thereof. In an effort to be clear, the protective layer may be referred to as the silicon carbide layer hereinafter, but the protective layer could be any one of the layers described above in other embodiments.

In an embodiment, the PS layer can include a macroporous/nanoporous hybrid framework. The nanopores are superimposed on the walls of the macropores. In an embodiment, the macropores can be about 0.5 to 20 μm deep and about 1 to 3 μm in diameter. In an embodiment, the nanopores can be about 1 to 20 nanometers in diameter.

In an embodiment, the contact can be disposed on and within the macroporous and nanoporous hybrid framework as well as extend above the PS layer and onto the protective layer (e.g., silicon carbide layer). In other words, the material fills in a portion of the PS layer and then forms a layer on top of the PS layer. The contacts are distinct and separated from one another by a space or area (e.g., a portion of the PS layer and a portion of the protective layer). In an embodiment, the contact can include one or more contact portions. In other words, one portion can be disposed on the PS layer and one portion disposed on the protective layer, but the two portions are contiguous in that a single metal layer extends from the PS layer onto the top of the PS layer and onto the protective layer. The contacts can be made of a metal or a combination of metals such as, for example, gold. In an embodiment, the contact includes a pre-coating layer usually titanium, and a metal layer usually gold, disposed onto the pre-coating layer. The pre-coating layer can be used to improve the electrical connection of the contact to the PS layer.

As mentioned above, the exposed portion of the PS layer not covered by contacts can have a plurality of nanostructures deposited (e.g., nanostructure islands) on and/or within the PS layer (e.g., a combined macroporous/nanoporous hybrid framework). The nanostructures can include, but are not limited to, a metal material, a metal oxide material, a metal oxynitride material, and combinations thereof. In an embodiment, the nanostructures can be discrete and/or clustered nanostructures and/or nanomaterials on and/or with the PS layer. In another embodiment, the nanostructures can be deposited onto and/or within discrete areas of the PS layer.

In an embodiment, the magnetic nanostructure can include a paramagnetic, ferrimagnetic, or ferromagnetic nanoparticle such as a nanosphere. In an embodiment, the magnetic nanostructure could be a nanowire, a nanodisk, or a nanobelt. In an embodiment, the magnetic nanoparticle can be uncoated or coated. In an embodiment, the magnetic nanostructure can include paramagnetic metals, or components with paramagnetic metals that are charged to create open shell configurations.

In an embodiment, the magnetic nanostructure can include metals and/or materials including these metals (as the metal charge distribution in a metal oxide or the like) including: tin, iron, nickel, titanium, cobalt, platinum, palladium, osmium, rhodium, ruthenium, molybdenum, aluminum, iridium, barium, calcium, cerium, dysprosium, erbium, europium, galodium, holmium, lithium, magnesium, manganese, molybdenum, samarium, sodium, strontium, thulium, tungsten, and zirconium, as well as metal oxides of each of these, or metal oxynitrides of each of these, where the specific magnetic nanostructures made from the oxides, oxynitrides has a magnetic property (e.g., paramagnetic, ferrimagnetic, or ferromagnetic property). In an embodiment, the metal oxide nanostructure can be converted to a metal oxynitride nanostructure in situ. In an embodiment, the nanostructure can be made of: tin oxynitride, iron oxide (Fe₂O₃, Fe₃O₄, or FeO), iron oxynitride, nickel oxide (NiO), nickel oxynitride, or cobalt oxide (Co₂O₃, Co₃O₄, or CoO), clustered oxides of each of these, other metal oxides, and a combination thereof, where the specific magnetic nanostructures made from the oxides, oxynitrides has a magnetic property (e.g., paramagnetic, ferrimagnetic, or ferromagnetic property). Using a variety of metal and metal oxide materials, the conductometric PS sensor can be designed to provide selectivity for a particular gas under a particular magnetic field.

In an embodiment, each nanostructure, a group of nanostructures, or a cluster of nanostructures, form an island on the PS layer. In an embodiment, the islands are spaced apart so there is no cross talk between the islands that would impede, substantially impede (e.g., impede about 50% or more, about 75% or more, about 90% or more, or about 50 to 90%, relative to no impedance), or substantially interfere (e.g., interfere about 80% or more, about 90% or more, or about 95% or more, relative to no interference) with the detection of the gas(es) of interest while subject to a magnetic field.

Embodiments of the conductometric PS sensor having a plurality of nanostructures on the PS layer can provide enhanced sensitivity and selectivity to certain gases while being subjected to a magnetic field. In particular, concentrations of select gases can be measured in the presence with one or more additional gases, where selected gases are more strongly sensed (e.g., impedance change detected). In an embodiment, the selectivity to one gas over one or more others can be controlled by selection of the type or combination of types of nanoparticles and magnetic field strength.

As briefly mentioned above, the conductometric PS sensor responds and is operative to measure an impedance change (e.g., an impedance magnitude change) across a first contact and a second contact that corresponds to a concentration of a gas in contact with the PS surface, while being subject to the magnetic field. The sensitivity of the conductometric PS sensor is defined as the relative increase or decrease in impedance over a time frame following exposure to a concentration of a gas of interest. It should also be noted that the sensitivity is, in part, a function of the analyte gas of interest, the nature of the nanostructure deposites, and whether the extrinsic semiconductor is n- or p-type.

The operating parameters of the conductometric PS sensor include, but are not limited to, a bias voltage, an AC voltage frequency, an AC voltage amplitude and combinations thereof. The conductometric PS sensors operate at a bias voltage between about 10 and 3000 millivolts DC. Also one can use an AC voltage frequency between 100 and 100,000 Hz, and an AC voltage amplitude between 1 and 1000 millivolts, or a combination thereof.

As mentioned briefly above, the impedance change can be measured with an impedance analyzer, a sensor and shunt circuit, or other impedance measurement devices. An embodiment of the sensor and shunt circuit uses a high impedance resistor in parallel with the sensor (conductometric PS sensor or components thereof). The resistor shunts the stray capacitance (removes high frequency noise), resulting in a resistive measurement.

The conductometric PS sensor can be used in a variety of ways including, but not limited to, a stand-alone detector device or system, or a device or system including an array of stand-alone detectors (e.g., one or more types of n-type or conductometric PS sensors and one or more types of p-type conductometric PS sensors), where in each instance the PS layer can be subject the magnetic field. The conductometric PS sensor can be used to detect gases (e.g., combustion generated gases such as carbon monoxide, carbon dioxide, sulfur dioxides, nitrogen oxides, and hydrogen sulfide) while subjecting the PS layer to a magnetic field. In particular, conductometric PS sensors, in accordance with the present disclosure, can provide a rapid and reversible response to analyte gases (e.g., including hydrogen chloride (HCl), ammonia (NH₃), phosphine (PH₃), carbon monoxide (CO), sulfur dioxide (SO₂), hydrogen sulfide (H₂S) nitric oxide (NOₓ), and toluene) at room temperature while being subjected to a magnetic field. Additional details regarding analyte gases are described in the Example.

In addition, the conductometric PS sensor can be used as an array, where multiple conductometric PS sensors (e.g., one or more types of n-type conductometric PS sensors and one or more types of p-type conductometric PS sensors) are uniquely sensitive to different gases of interest thereby enabling an array to measure the concentration of multiple gases simultaneously (e.g., an appropriately treated conductometric PS sensor can be made to respond more strongly to one gas over a second gas), where each PS sensor can include its own magnetic field source or use a commone magnetic system to apply the magnetic field. In addition, an array of conductometric PS sensors can be used to enhance sensing selectivity as the array of conductometric PS sensors provide multiple data points per tested sample which can be analyzed in a matrix format to provide selectivity for one gas over another based on the individual conductometric PS sensors within the conductometric PS sensor array. Thus, an array of conductometric PS sensors includes conductometric PS sensors sensitive to one gas over another and, in this sense, to select gases. In this regard, the array of conductometric PS sensors can be used to detect multiple analytes simultaneously.

Embodiments of the present disclosure also include methods of making conductometric PS sensors. In general, the conductometric PS sensor can be fabricated by first providing a silicon substrate (or other appropriate substrate) having a protective layer, such as a silicon carbide layer, disposed on a first portion of the silicon substrate. Then, a first area on the silicon substrate is converted into a PS layer, where the first area does not have a silicon carbide layer disposed thereon. Next, a first contact (e.g., a first contact pre-coating (e.g., Ti or Cr) and the first contact (e.g., Au)) is formed onto a first portion of the PS layer and onto a first portion of the silicon carbide layer. The first portion of the silicon carbide layer is contiguous with the first portion of the PS layer as described above. A second contact (e.g., a second contact pre-coating (e.g., Ti or Cr) and the second contact (e.g., Au)) is formed onto a second portion of the PS layer and onto a second portion of the silicon carbide layer. The second portion of the silicon carbide layer is contiguous with the second portion of the PS layer as described above. A third portion of the PS layer is between the contacted first portion and the second portion of the PS layer. The first and second contacts can be formed at the same time. Specifically, the first and the second contact pre-coatings are formed and then the first and second contacts are formed on the first and second contact pre-coatings, respectively. The first and second contact pre-coatings are advantageous in that the first and second contacts enable a superior electrical connection to be formed. In addition, the first and second contacts can be formed using a shadow mask technique. One or more types of nanostructures can be fractionally disposed on the PS layer, where the nanostructure(s) form islands on the PS layer.

Additional fabrication steps can be conducted. For example, an additional fabrication step includes cleaning the PS layer with a mixture of one part hydrochloric acid (e.g., about 44%) in about five parts methanol for about four hours. In addition, a fabrication step for forming a nanostructured deposit on the porous silicon layer can be performed.

In an embodiment, the silicon substrate can be replaced with any extrinsic semiconductor (e.g., GaP, InP, CdTe, and the like) onto which a porous microstructure can be generated. In an embodiment, the silicon substrate can include wafers, such as, but not limited to, p- or p⁺-type doped wafers, n- or n⁺-type doped wafers, n- or n⁺-type phosphorous doped wafers. In an embodiment, the silicon substrate can include wafers, such as, but not limited to, p- or p⁺-type doped silicon wafers, n- or n⁺-type doped silicon wafers, n- or n⁺-type phosphorous doped silicon wafers.

In an embodiment, other materials can be used in place of the silicon carbide layer such as, but not limited to, a silicon nitride layer, a SiOₓN_{y} layer, an insulating dielectric film, and a ceramic layer.

In an embodiment, the PS layer can be prepared by electrochemically etching a portion of the silicon substrate with acetonitrile, hydrofluoric acid, tetrabutylammonium-perchlorate (TBAP), and water, for example. Additional details regarding the preparation of the n-type PS layer are presented in more detail herein.

In an embodiment, the first contact pre-coating layer and the second contact pre-coating layer can be made from titanium or chromium, for example, and can be about 100 to 300 angstroms thick or about 200 angstroms thick. The first contact pre-coating and the second contact pre-coating can be disposed onto the two portions of the PS substrate via techniques such as, but not limited to, electron-beam evaporation, sputtering, electroless plating, and electroplating.

In an embodiment, the first and second contacts and can be made of metals, such as, but not limited to, gold (Au), and can be about 1000 to 4000 angstroms thick or about 3000 to 5000 angstroms thick to facilitate wire bonding. The first contact and the second contact can be disposed onto the two portions of the PS substrate via techniques such as, but not limited to, electron-beam evaporation, sputtering, electroless plating, and electroplating.

In an embodiment, the nanostructures can be can be disposed using techniques such, but not limited to, electron-beam evaporation, sputtering, silk-screen printing, electroless plating, and electroplating.

After the conductometric PS sensor is formed, the conductometric PS sensor can be validated. In this regard, embodiments of this disclosure include methods of selecting a conductometric PS sensor having certain performance characteristics, methods of analyzing the data measured using the conductometric PS sensor, and methods of measuring the concentration of a gas. In addition, the method of validating includes detecting false positives (e.g., determining that an impedance change is from the gas of interest and not a response caused by another source). Furthermore, the present disclosure provides methods of analyzing data for the conductometric PS sensor as well as for other devices and sensors.

### Examples

Now having described the embodiments of the present disclosure, in general, the following examples describe some additional embodiments of the present disclosure. While embodiments of the present disclosure are described in connection with the examples and the corresponding text and figures, there is no intent to limit embodiments of the present disclosure to this description. On the contrary, the intent is to cover all alternatives, modifications, and equivalents included within the spirit and scope of the embodiments of the present disclosure.

### Example 1

### Brief Introduction:

Small magnetic fields are found to greatly enhance the reversible room temperature conductometric responses of n and p- type porous silicon (PS) interfaces, treated with nanostructured island sites containing paramagnetic Co(II) and Fe(II). At concentrations sufficiently low so as to avoid cross talk between the nanostructured island sites, the response to NO concentrations demonstrates the significant effect which the Co(II) and Fe(II) have on the decorated extrinsic semiconductor majority charge carriers as they direct a dominant electron transduction process for reversible electron transduction and chemical sensing (IHSAB principle) in the absence of significant chemical bond formation. Co(II) and Fe(II) oxide sites enhance response and provide a means for small magnetic fields to interact with and enhance the sensor interface response. For p-type systems, the interaction is with small virtually constant thermal electron populations lying above the Fermi energy at 0° K. The electron removal rate increases with magnetic field strength. At the highest magnetic fields and NO analyte concentrations the available electron population is depleted, and the response to the analyte decreases at higher concentrations. At lower magnetic fields (<1000 G) the response faithfully follows concentration. For n-type systems, the magnetic field interaction increases increases resistance. This increase in response may be attributed to the interaction with donor levels ˜0.025 eV below the conduction band. A substantial enhancement of sensor response relative to that for the Co(II) and Fe(II) treated PS interfaces is observed, with the introduction of a small magnetic field greatly increasing an already enhanced conductometric response.

### Introduction:

We have previously outlined' an approach to “nanostructure directed electron transduction-physisorption vs. chemisorption” on a sensor/microreactor interface. The gas analyte, acting as a Lewis base, donates electrons to the porous silicon semiconductor surface. For p-type semiconductors, the majority charge carriers, electron holes, combine with the donated electrons. Thus the majority charge carrier population decreases leading to an increase in resistance. In contrast, for n-type semiconductors the majority charge carriers are electrons, and so the majority charge carrier population increases leading to a decrease in resistance. We employ the fractional deposition of nanostructured island sites to modify highly sensitive surface layers created utilizing a hybrid nanopore covered microporous matrix formed on “p- or n-type” silicon. The procedure relies on the correlation of the tenants of acid/base interaction and the properties of extrinsic semiconductors^{1,2,3}. At the core of the concept is the Inverse Hard and Soft acid/base interaction model (IHSAB), which can provide a general approach to optimally design sensors and microreactors with improved and variable sensitivity and conversion efficiency for a variety of gases in an array-based format¹⁻⁴. In the present study, we demonstrate how small magnetic fields influence and enhance the variable, complimentary, and distinct nature of nanostructure decorated p and n-type silicon within the IHSAB concept⁵.

The IHSAB principle/approach, facilitates a dominant electron transduction and complements the HSAB principle for hard/soft acid/base interactions first espoused by Pearson et al.⁶ and later correlated with density functional theory (DFT)^{7,8} and Chemical Reaction Theory⁹ by Pearson, Parr^{7,8}, and Cohen et al.⁹. The HSAB principle dictates that soft-soft acid/base interactions produce significant covalent bonding and hard-hard combinations produce significant ionic bonding and that hard acids prefer to coordinate to hard bases and soft acids prefer to coordinate to soft bases. In contrast, the driving principle to promote a dominant electron transduction represents the inverse (IHSAB) of that necessary to form strong chemical bonds². This driving force is manifest through the introduction and interaction of metal oxide nanostructured islands, which strongly influence the nature of electron transfer involving the majority charge carriers in a p or n-type semiconductor.

The IHSAB concept facilitates designed, highly variable, surface interactions using an available diversity of nanostructured “fractional” oxide depositions to focus the nature of the surface-interface interaction while minimizing the chemical interaction of an acidic or basic analyte with the semiconductor interface. To this framework, we now introduce magnetic nanoparticles in order to gauge their interaction with magnetic fields. This initial study makes use of the Co(II)Cl₂.6H₂O complex¹⁰ so as to deposit Co(II) in the pores of a porous silicon matrix^{5,11} and 10 nm iron oxide nanoparticles so as to deposit Fe(II) in the pores of the PS matrix^{5,11}.

In this study, we make use of the fact that an analyte can donate electrons to a “p-type” PS semiconductor surface and these electrons combine with holes, thus reducing the number of majority charge carriers. This leads to an increased resistance. The process is reversed for an “n-type” semiconductor as the majority charge carriers, electrons, increase and the resistance decreases. Here we expand on the predictions of the IHSAB concept, as it promotes electron transduction vs. chemical bond formation and can be applied in concert with, and in complement to, the behavior of an extrinsic semiconductor.

The conductometric technique used in this study is extremely sensitive. Typical X-ray photoelectron spectroscopy (XPS) spectra are sensitive to ˜0.1%. The typical time frame for the depositions used to obtain the conductometric data is 30 seconds. XPS data requires that we use deposition times, which are at least 5 minutes. This corresponds approximately to an order of magnitude increase in concentration to obtain signals in the 0.1% range. The selection of the nanostructures and the variable surface sensitivities that are produced as they form in-situ metal oxide deposits, introduces a distinct systematics of design. The nanostructure deposition must be maintained at a sufficiently low level to avoid cross talk between the nanostructures that, as it increases, leads to a noisy device and the eventual loss of functionality. In combination, these can be used as a basis to develop selectivity. We emphasize that establishing the optimum deposition requires a careful tuning of the decorating concentrations.

### Experimental

As described earlier^{1,11}, to create the framework to develop highly efficient nanostructure modified interfaces on either p- or n-type PS, a micro/nanoporous interface must be generated'. Here, an array of micrometer diameter pores, all of which have nanometer diameter porous walls, are etched into a silicon wafer. Schematic diagrams of the complete working sensor platform have already been presented^{1,4,11,12.} The PS interface is generated by electrochemical anodization of 1-20 Ohm-cm, n-type (phosphorous doped) (100) silicon wafers (Wafer World), 7-13 Ohm-cm, p-type (boron doped) (100) silicon wafers (Siltronix), and 1-3 Ohm-cm p-type (denoted p⁺) (boron doped) (100) silicon wafers (Siltronix). The selected doping range for these systems was chosen to insure that a nanopore covered microporous hybrid structure could be generated¹³. The anodization for the p-type wafers is done as they are etched in 1M HF, and 0.1 M tetrabutylammonium perchlorate (TBAP) in acetonitrile (MeCN) at 3-6 mA/cm². The anodized sample is cleaned in MeCN for 10 minutes to purge any residue in the pores due to the etch solution^{1,4,11,12.} Subsequently, the sample is immersed for several minutes in HF and then methanol. The PS has a porosity of 50-80% with the μpore diameters varying from 0.8 to 1.5 μm and pore depths varying from 10 to 30 μm. The anodization for the p⁺-type wafers is done in 4M HF and 4.8M water with the remaining solution being DMF. The etch was performed at 10 mA/cm² for a period of 10 minutes¹⁴. After the etch, the sample is soaked in 0.1M KOH until the evolution of gas bubbles has ceased¹⁴. The anodization of the n-type wafers^{14,15} is done under topside illumination using a Blak-Ray mercury lamp. The wafer is etched in a 1:1 solution of HF and ethanol at a current between 8-15 mA/cm² (FIG. 2(a))^{1,13}. A prepared n-type anodized sample is placed in methanol for a short period and then transferred to a dilute HF solution for a 30-minute period. This process creates a porous structure with pore diameters of order 0.5-0.7 μm and pore depths varying from 50 to 75 μm.

As we have previously noted^{1,4,11,12,} before the anodizations, an insulation layer of SiC (≈1000 Angstroms) is coated onto the crystalline silicon (c-Si) substrate by plasma-enhanced chemical vapor deposition (PECVD) methods and windows of 2×5 mm are opened in this layer by Reactive Ion Etching (RIE). The SiC layer serves two purposes. SiC makes it possible to form the hybrid micro/nanoporous PS structure in the specified windows during electrochemical anodization because of its resistance to HF. The SiC also aids the placement of gold contacts exclusively on the interfaces. We continue to employ low resistance gold contacts whose formation has been discussed in detail previously^{4,3}. The PS hybrid arrays of nanopore-covered micropores are tested at room temperature for their individual sensor response. In the present study, the selected fractional nanostructure deposition is used to create an improved physisorption dominated response. The nature of this response is based on the use of the IHSAB acid/base principle¹⁶.

In this study we treat the micro-/nanoporous framework with CoCl₂.6H₂O at various initial concentrations and degrees of water removal, measuring the effect of a magnetic field on the sensor response of the decorated interface. For comparison, we also treat the interface with nanotitania¹⁷. We are not applying a coating technique that requires an exacting structural film arrangement but a much simpler process. In order to maintain a sensor response consistent with the IHSAB principle, the nanostructure deposition must be maintained at a sufficiently low level to avoid cross-talk between the nanostructures¹¹. Selected nanostructured metals, metal oxides, and nanoparticle catalysts can be deposited to the nanopore-covered micropores to provide for distinct and variable sensitivities¹¹. Following this procedure, we have also treated the micro-/nanoporous PS interface with 10 nm iron oxide particles functionalized with NH₃.

Based on previous results¹⁸, nanostructured deposits of cobalt(II) chloride hexahydrate are partially oxidized as the prepared sensor interfaces are subsequently cleaned and as the sensors are tested at atmospheric pressure . The initially introduced titania (anatase) may be crystalline, however, we cannot be certain of this crystallinity after deposition to the PS surface. The untreated PS hybrid structures are exposed for 30 seconds to the CoCl₂.6H₂O at varying concentrations or to nanotitania solutions and are subsequently placed in deionized (DI) H₂O and MeOH for consecutive 120-second periods. At the lower cobalt concentrations, the conductometric response agrees well with that expected from a well-behaved conductometric sensor response, however, the introduction of a magnetic field diminishes this response. As the cobalt concentration is increased, the sensor response decreases from that of the untreated PS interface but can be enhanced substantially through introduction of a magnetic field¹⁹.

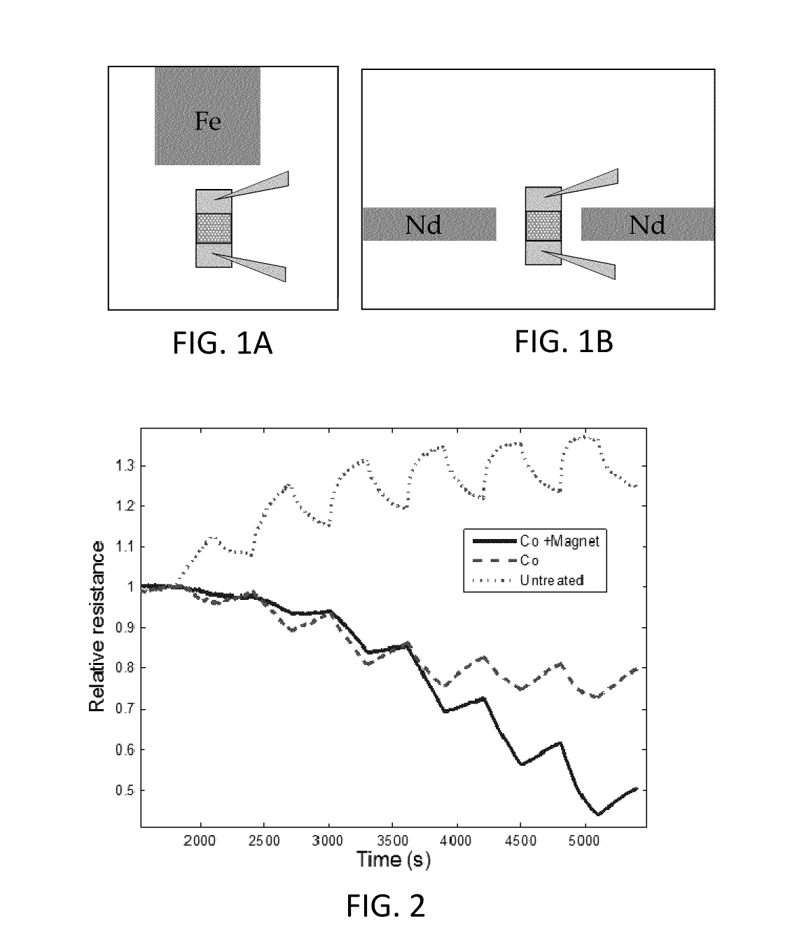

The magnetic fields used in these experiments vary from 80 to 2000 Gauss (0.008-0.2 Tesla). They are generated using the two configurations pictured in FIGS. 1(a) and 1(b). In the first configuration, a classical iron magnet creates a field of 80 Gauss at the sensor interface. The second configuration uses a neodymium rare earth (nickel coated) magnet placed so as to create a magnetic field of ˜200 or 2000 Gauss at the sensor interface. The magnitudes of these fields at the sensor are measured with a Lakeshore Model 421 Gaussmeter at the location of the sensor.

These systems are evaluated in an unsaturated mode as the conductometric response for NO is measured. This is useful because the time scale for reversibility may become an issue in a long term saturated mode. Further, the longer-term exposures are not necessary. However, the sensor response and recovery times are distinctly different. This means that full time recovery from the gas exposure takes longer than 300 s, the initial exposure time duration in the present configuration (also see Ref 10). Nevertheless, the onset of the sensor response for these atmospheric pressure open inlet studies^{3a,4} remains clearly detectable. This behavior suggests that the responses for the free radical, NO on PS are that of a gas whose interaction is dominated by physisorption but which also displays weak chemisorption^{4,12}. If the sensor surface is purged with ultra high purity (UHP) N₂ for longer durations a gradual shift to the initial base line is observed. The return to baseline can be further improved by more tightly constraining the gas flow path to the sensor surface.

In all cases, the analyte gas being sensed is brought to the hybrid surface after entrainment in UHP nitrogen (Matheson 99.999+%) at room temperature. The system is purged for a minimum of 30 minutes before use with this UHP nitrogen, which provides for the removal of residual water¹². The typical resistances for the base PS structures range between 300 and 10,000 Ohms at room temperature.

### RESULTS

FIG. 2 depicts results obtained as CoCl₂.6H₂O from a solution at a concentration of 1.19 g/10 ml=0.5 molar is deposited to an n-type PS interface and dried in air. Amphoteric NO in the range 1-10 ppm, entrained in UHP N₂ and brought to the PS interface produces a conductometric signal corresponding to an increase in resistance and is indicative of the interaction of an acid gas^{5,11}. Treatment with the CoCl₂.6H₂O , which is subsequently dried in air, as indicated in the experimental section, produces a surface, which overcomes the weak acidity of NO leading to the extraction of electrons from this gas and a decrease in resistance as a function of analyte concentration. Here, the Co(II) compound, deposited at a concentration to avoid cross-talk between the nanostructures, acts as a stronger acid than NO so as to extract electrons. At this cobalt concentration, the introduction of an 80 Gauss magnetic field produces an enhanced conductometric response, which is a function of analyte concentration and corresponds to a decrease in resistance with magnetic field.

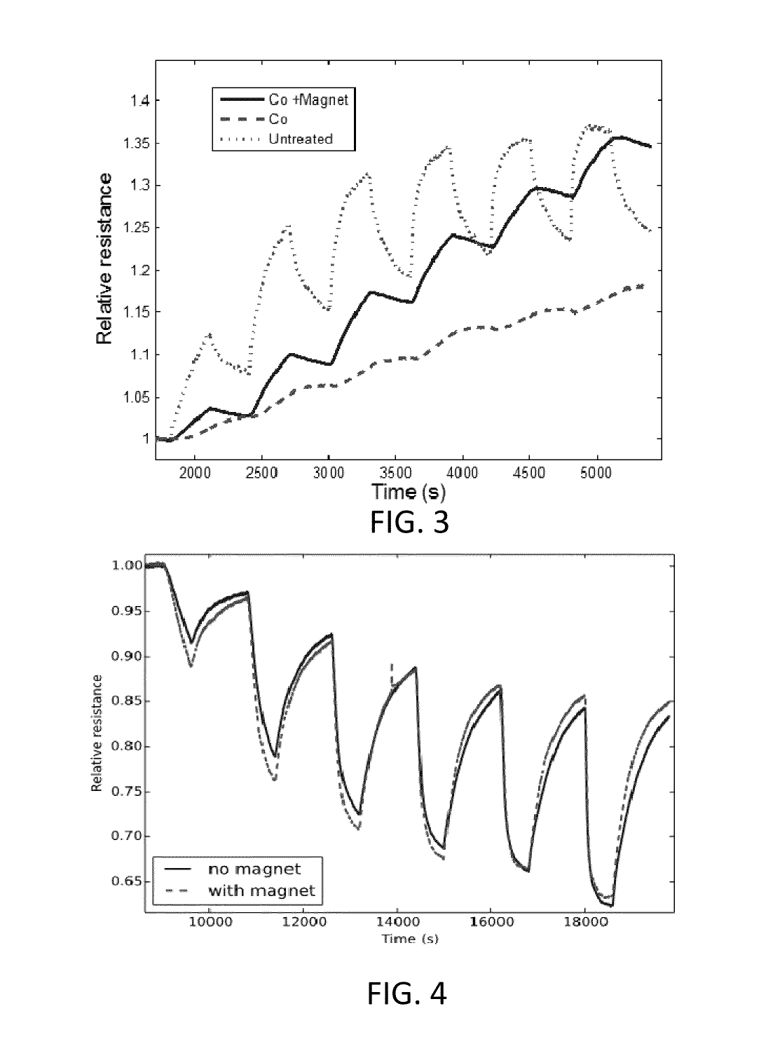

FIG. 3 depicts results obtained as CoCl₂.6H₂O from a solution at a concentration of 0.5 molar is deposited to an n-type PS interface and heated to a temperature of 50° C. to remove a significant component of water and form a Co(II)Cl₂ chain, accompanied by a decreased water concentration¹⁰. This leads to a water depleted long chain of CoCl₂ molecules surrounded on top and bottom by water molecules¹⁰. Amphoteric NO in the range 1-10 ppm, entrained in UHP N₂ and brought to the PS interface produces a conductometric signal corresponding to an increase in resistance and indicative of the interaction of an acid gas¹⁰. Now treatment with the resulting CoCl₂ hydrated polymer produces a surface that does not overcome the weak acidity of NO. The Co(II) deposition leads to a decreased signal corresponding to a considerable decrease in the extraction of electrons by the NO and a decrease in the resistance as a function of NO concentration. Here, the Co(II) compound again counters the extraction of electrons by NO. At this cobalt concentration, the introduction of an 80 Gauss magnetic field produces an enhanced conductometric response relative to the Co(II) deposited surface and corresponding to an increase in resistance with analyte concentration and magnetic field.

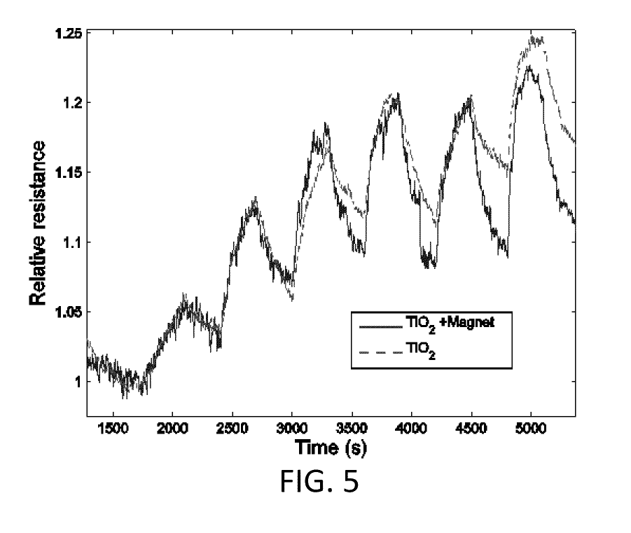

By comparison, FIG. 4 reveals no effect by the magnetic field on untreated porous silicon. FIG. 5 indicates the response after nanotitania, at a concentration limited to a value to avoid cross talk between the nanostructures, is deposited to an n-type PS interface. Amphoteric NO in the range 1-10 ppm, entrained in UHP N₂ and brought to the PS interface produces a conductometric signal corresponding to an increase in resistance with analyte concentration and indicative of the interaction of an acid gas^{5,11} with an interface that has been mildly treated with TiO₂. Treatment after the TiO₂ deposition to the PS interface produces a surface, which does not overcome the weak acidity of NO. Of greater importance is the fact that the signal observed with a magnetic field (up to 2000 Gauss) for this non-magnetic material is virtually identical to that of the untreated interface. While there is clear concentration dependence with analyte, the non-magnetic nanostructured TiO₂ deposition is clearly not affected by the presence of the magnetic field.

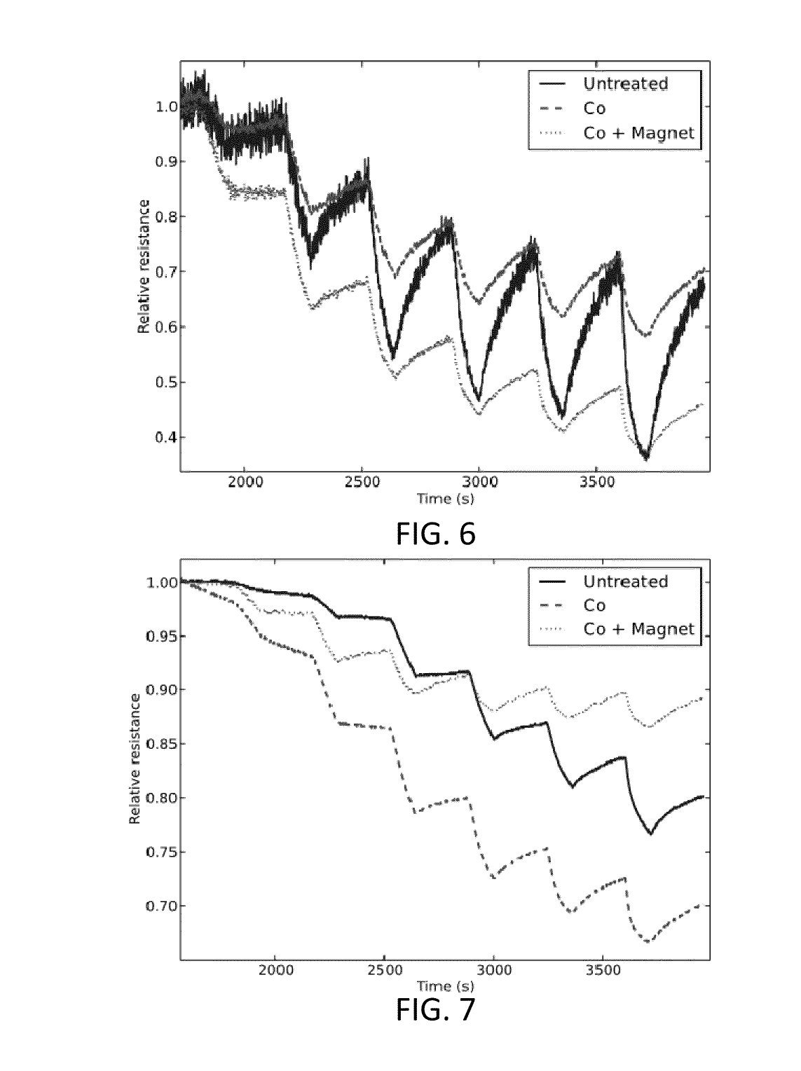

FIG. 6 depicts results obtained as CoCl₂.6H₂O at a solution concentration of 0.5 molar is deposited to a p-type PS interface. Amphoteric NO in the range 1-10 ppm, entrained in UHP N₂ and brought to the PS interface produces a conductometric signal corresponding to a decrease in resistance and indicative of the interaction of an acid gas^{5,11} as it removes electrons from a p-type PS surface. Treatment with the CoCl₂.6H₂O produces a surface, which quenches the effect of the weak acidity of NO leading to a decrease in the extraction of electrons by this gas and a decrease in the conductance as a function of analyte concentration. This change likely results as the concentration of CoCl₂.6H₂O is sufficient to force cross talk between the nanostructures, hence a decrease in conductance. At this cobalt concentration, the introduction of an 80 Gauss magnetic field produces an enhanced conductometric response corresponding to a decrease in resistance (increase in conductance) with magnetic field and analyte concentration.

FIG. 7 depicts results obtained as CoCl₂.6H₂O at a solution concentration of 0.25 molar, half that of FIG. 6, is deposited to a p-type PS interface. Amphoteric NO in the range 1-10 ppm, entrained in UHP N₂ and brought to the PS interface produces a conductometric signal corresponding to an increase in conductance with analyte concentration and indicative of the interaction of an acid gas⁵⁻¹¹ as it removes electrons from a p-type PS surface. Treatment with the CoCl₂.6H₂O produces a surface that enhances the effect of the weak acidity of NO leading to an increase in the extraction of electrons by this gas and an increase in the conductance as a function of concentration. This result is consistent with the expected enhancement of the conductometric conductance due to CoCl₂.6H₂O at a concentration sufficiently low so as to prevent cross talk between the decorated nanostructures and hence an increase in the conductance. The result is in agreement with the recently developed IHSAB principle. At this cobalt concentration, the introduction of a ˜200 Gauss magnetic field does not interact so as to increase the conductometric response. While there appears to be some increase relative to the PS interface at 1-3 ppm, this increase does not match that observed for the Co(II) deposition. The signal is also constant at pressures over the range 3 to 10 ppm where the magnetic field no longer appears to follow the analyte concentration increase.

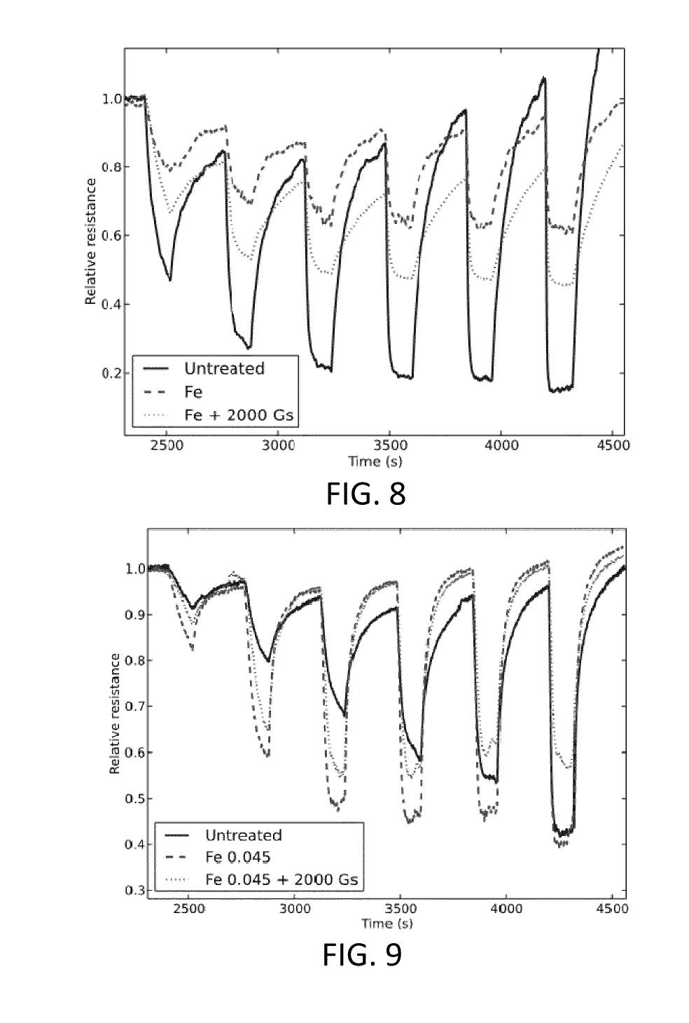

FIG. 8 depicts results obtained as 10 nm iron oxide Fe(II) nanoparticles functionalized with NH₃ are deposited to a p-type PS interface at a concentration of 0.05 (see exptl. section). Amphoteric NO in the range 1-10 ppm, entrained in UHP N₂ and brought to the PS interface produces a conductometric signal corresponding to a decrease in resistance with analyte concentration and indicative of the interaction of an acid gas⁵⁻¹¹ as it removes electrons from a p-type PS surface. Treatment with the iron oxide nanostructures produces a surface which greatly quenches the response due to the weak acidity of NO and leads to a decrease in the extraction of electrons by this gas and, hence, to a decrease in the conductance as a function of concentration. This change likely results as the concentration of iron oxide nanostructures facilitates cross talk between the nanostructures and hence to⁵⁻¹¹ a drop in conductance. Further, it is apparent that the ratio of intensities at NO concentrations greater than 3 ppm is severely curtailed (quenched in intensity). At these Fe(II) concentrations, the introduction of a 2000 Gauss magnetic field produces an enhanced conductometric response relative to that obtained from the Fe(II) deposition, corresponding to a decrease in resistance (increase in conductance) with magnetic field. However, this response is considerably less than that for the PS interface.

FIG. 9 depicts results obtained as 10 nm iron oxide Fe(II) nanoparticles functionalized with NH₃ at a lowered concentration (0.045—see exptl.) relative to FIG. 8 are deposited to a p-type PS interface. Amphoteric NO in the range 1-10 ppm, entrained in UHP N₂ and brought to the PS interface produces a conductometric signal corresponding to a decrease in resistance with analyte concentration and indicative of the interaction of an acid gas⁵⁻¹¹ as it removes electrons from a p-type PS surface and thus increases conductance. Treatment with the iron oxide nanostructures produces a surface which greatly enhances the response of the PS interface to the weakly acidic NO, leads to an increased extraction of electrons by this gas and, hence, an increase in the conductance (hole carriers) as a function of analyte concentration. This change results as the concentration of iron oxide nanostructures is sufficiently low so as to avoid cross talk between the nanostructures. The substantial increase in conductance is expected from the IHSAB principle Further, the response follows the concentration up to levels of 4 ppm NO, but begins to quench at NO concentrations in excess of 5 ppm. The response, is not only considerably greater than that in FIG. 8, but far less noisy. At the deposited Fe(II) concentrations, the introduction of a 2000 Gauss magnetic field produces a response that parallels that of Fe(II) at the lowest concentrations but then shows a considerable quenching relative to both the response of the PS interface and the Fe(II) decorated interface. This degree of quenching suggests that limited available electron concentration (above the 0° K. Fermi level-see following) at the temperature of the experiment is being depleted at a significantly enhanced rate from the decorated PS interface through magnetic field interaction. This distinguishing characteristic is at 2000 Gauss, which suggests a comparison with lower magnetic fields.

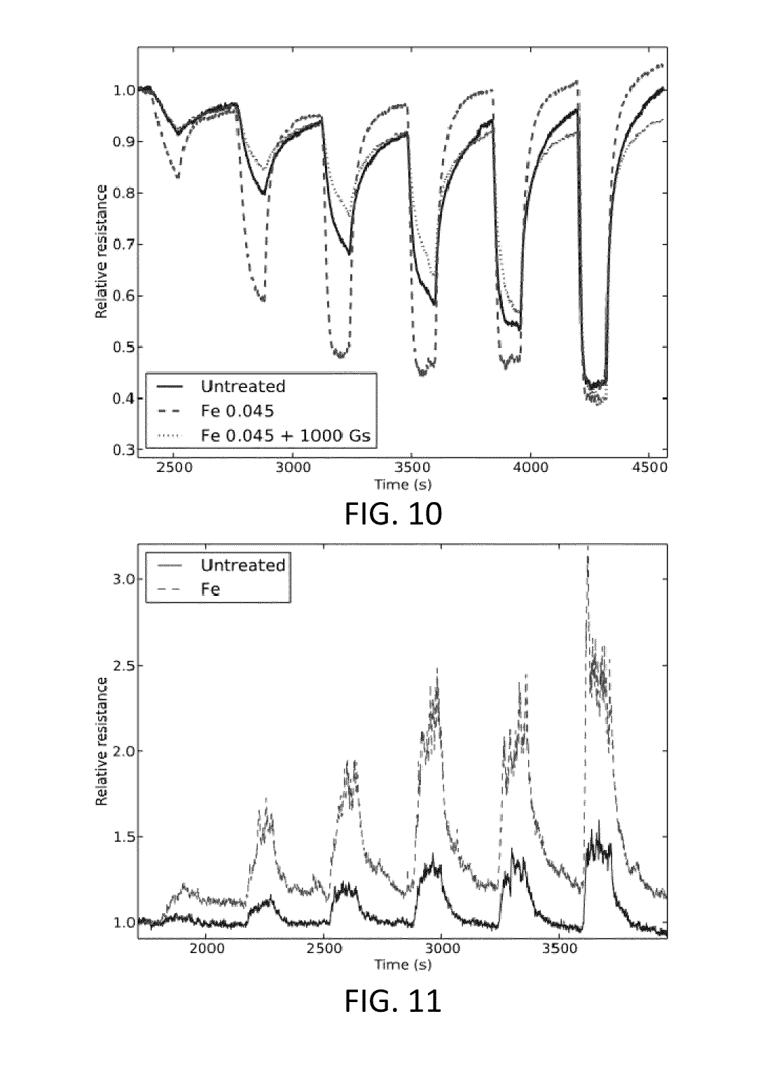

FIG. 10 depicts results obtained as 10 nm iron oxide Fe(II) nanoparticles functionalized with NH₃, again at a lowered concentration (0.045—see exptl.) relative to FIG. 8, are deposited to a p-type PS interface. Again, amphoteric NO in the range 1-10 ppm, entrained in UHP N₂ and brought to the PS interface to produce a conductometric signal corresponding to a decrease in resistance with analyte concentration and indicative of the interaction of an acid gas^{5,11} as it removes electrons from a p-type PS surface. Treatment with the iron oxide nanostructures produces a surface which greatly enhances the response due to the weak acidity of NO and leads to an increase in the extraction of electrons by this gas and, hence, an increase in the conductance as a function of analyte concentration. Again, the concentration of iron oxide nanostructures is sufficiently low so as to avoid cross talk between the nanostructures, leading to a substantial increase in conductance as expected from the IHSAB principle. However, in sharp contrast to FIG. 9, at the deposited Fe(II) concentrations, the introduction of a 1000 Gauss magnetic field produces a response that, while somewhat weaker, faithfully follows concentration through 5 ppm, and is comparable to that at 10 ppm to that of both the Fe(II) decorated and untreated PS interface. It is apparent that the electron concentration at the PS surface is depleted at a much slower rate than that characteristic of the response recorded for the 2000 Gauss field. The response variation as a function of analyte concentration suggests that the thermally populated electrons with which the magnetic field interacts are being depleted at a significantly slower rate from the decorated PS interface through magnetic field interaction. This suggests an inherent limit to the electron population with which the magnetic field is interacting dictated by thermal excitation above the Fermi level, E_{F}. These loosely bound electrons interact with the magnetic field and are depleted at a rate proportional to the magnetic field thus increasing the concentration of holes and p-type interface conductance.

FIG. 11 depicts results obtained as 10 nm iron oxide Fe(II) nanoparticles functionalized with NH₃ at a lowered concentration (0.040—see exptl.) are deposited to an n-type PS interface. Amphoteric NO, in the range 1-10 ppm, is entrained in UHP N₂ and brought to the PS interface to produce a conductometric signal corresponding to an increase in resistance and indicative of the interaction of an acid gas⁵⁻¹¹ as it removes electrons from the n-type PS surface. Treatment with the iron oxide nanostructures produces a surface which greatly enhances the response to NO and leads to an increase in the extraction of electrons by this gas and an increase in the response as a function of concentration. This corresponds to an enhanced removal of electrons from the donor level. The change results as the concentration of iron oxide nanostructures is sufficiently low so as to avoid cross talk between the nanostructures. We now observe a substantial increase in conductance as a function of analyte concentration as expected from the IHSAB principle. The response follows concentration up to concentrations of 4 ppm NO, beginning to quench at 5 ppm.

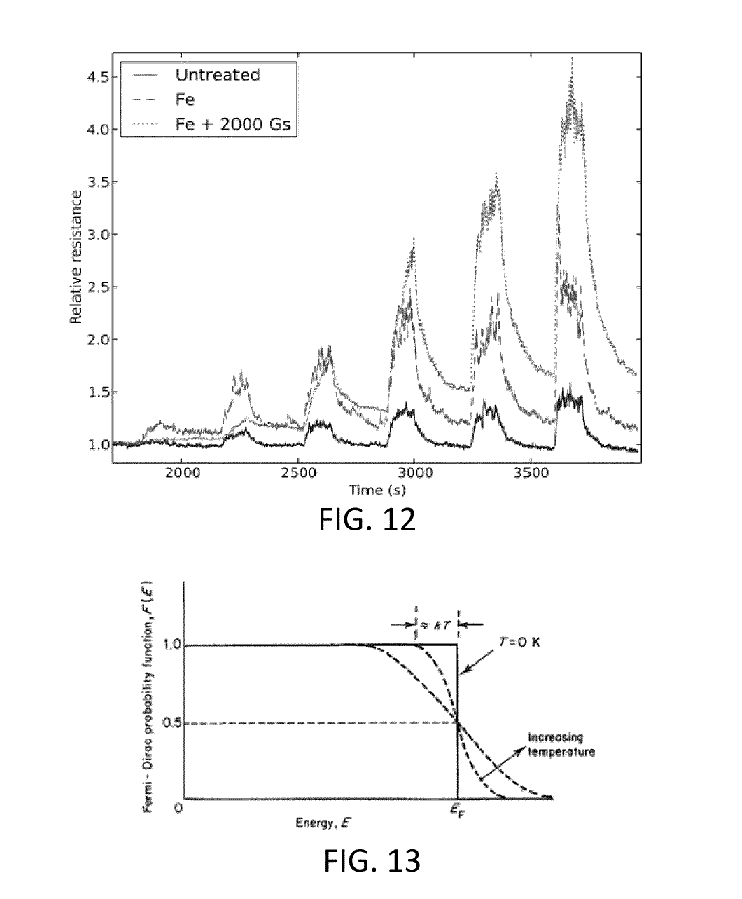

FIG. 12 depicts results obtained as 10 nm iron oxide Fe(II) nanoparticles functionalized with NH₃ at a lowered concentration (0.040—see exptl.) are deposited to an n-type PS interface. Amphoteric NO in the range 1-10 ppm, entrained in UHP N₂ and brought to the PS interface to produce a conductometric signal corresponding to an increase in resistance as a function of analyte concentration and indicative of the interaction of an acid gas^{5,11} as it removes electrons from the n-type PS surface. Treatment with the iron oxide nanostructures produces a surface which greatly enhances the response due to NO and leads to an increase in the extraction of electrons by this gas and, hence, an increase in the resistance as a function of concentration. This change results as the concentration of iron oxide nanostructures is sufficiently low so as to avoid cross talk between the nanostructures. We again observe a substantial increase in resistance as expected from the IHSAB principle. Further, one notes that the response follows concentration up to concentrations of 4 ppm NO, beginning to quench at 5 ppm. At the deposited Fe(II) concentrations, the introduction of a 2000 Gauss magnetic field produces a response that closely parallels that of Fe(II) at the lowest concentrations but then shows a considerable increase relative to both the response of the PS interface and the Fe(II) decorated interface. This increase suggests the possibility that the electrons with which the magnetic field interacts are being removed from the donor levels at an increased rate. The electron removal will be suggested to go through the conduction band for the n-type semiconductor. There is a considerable increase in the response of the decorated PS interface through magnetic field interaction. This behavior is again significant.

### DISCUSSION

We suggest that the discussion of these systems should begin with the consideration of the Fermi-Dirac level distribution and its correlation with the energy levels of extrinsic semiconductors depicted in FIG. 13²⁰. Both p and n type semiconductors have similar thermal distributions. We are concerned with the deviation of this distribution at temperatures greater than 0° K. as those electrons at energies above E_{F} in FIG. 13 can readily interact with a magnetic field. These electrons have thermal temperatures, kT˜208 cm⁻¹=0.025 eV at room temperature. It is unlikely that an electron will be excited at room temperature if it lies more than 0.1 eV below the Fermi level as the states within an energy range of kT are almost entirely filled²⁰. This suggests that in a p-type semiconductor (see below), the easily accessed electron population is small and virtually at a constant value at a given temperature. As a magnetic field interacts with those electrons that are easily accessible, the rate of interaction and electron removal increases with magnetic field. This should produce a signal at the lowest NO concentrations that becomes muted at the higher NO concentrations as the electron population is depleted. The effect should be more pronounced the higher the magnetic field and lead to a notable quenching at higher NO concentrations. We suggest that this effect is manifest in the response to the 2000 Gauss magnetic field in FIG. 9. At lower magnetic field strengths, the available electron population should be depleted more slowly. The response to the 1000 Gauss magnetic field in FIG. 10, that is initially weaker, follows the NO concentration dutifully through 5 ppm and displays a significant intensity at 10 ppm. We suggest that this results as the virtually constant electron population above E_{F} at a given temperature is not as rapidly depleted. The results we observe for the iron oxide system in FIG. 9 also appear to be manifest in the Co(II) results of FIG. 7.

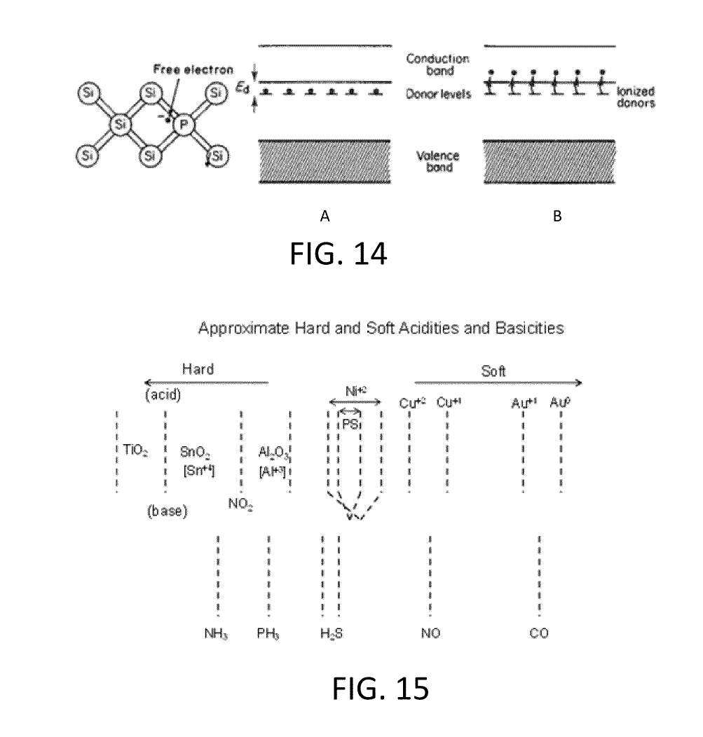

In contrast, the n-type semiconductor framework with free electrons readily accessible from the donor levels, within E_{d}˜0.025 eV of the conduction band, furnishes a significant population over and above the thermal level population (FIG. 14) (This is in contrast to the acceptor level population lying within 0.01 eV of the valance band and more than 1 eV below the conduction band for a p-type system).

The data in FIGS. 11 and 12 demonstrate the significant affect which the fractional Fe(II) population has as it leads to an increase in sensitivity as dictated by the IHSAB principle. FIG. 12 demonstrates that the introduction of a small magnetic field leads to a further considerable increase in the response of the interface. This increase might be explained with the use of FIG. 14. The excess electrons in the n-type system are less tightly bound and should be more accessible to the magnetic field than are other sites in the lattice. Calculations demonstrate that the applied magnetic fields correspond to an energy increment that is of same order of magnitude or slightly larger than the 0.025 eV donor level-conduction band separation. Alternatively, the introduction of these magnetic fields should allow one to further remove electrons from the donor level to the conduction band thus decreasing the donor charge carriers and increasing the response of the system to NO (resistance increase). This equivalent electric field, 0.01-0.025 eV, exceeds by several orders of magnitude that which would be obtained from Eₚₑᵣₚ associated with the Hall effect.^{21,22} The magnitude of the Hall effect in these systems is sufficiently small that it must be measured in an AC mode and is not accessible to DC measurement.

The small magnetic fields necessary to significantly enhance the sensor response for the Co(II) and Fe(II) systems we have studied make use of the fundamental properties of an extrinsic semiconductor as they are coupled to an easily deposited and controllable interface. These results suggest the viability of small devices in which the analog of small coils placed in close proximity to the Co(II) and Fe(II) decorated interfaces will be amenable to the readily measureable enhancement of sensor response.

We have obtained response data for several metal oxides which we have found to be predicted by the IHSAB concept^{5,11,16,23,24} forming “materials sensitivity matrices” for a given analyte as outlined in FIG. 15. This will enhance the capability to sense analytes and their mixtures. In FIG. 15, the analyte scale is fixed in terms of acid/base properties, as determined by the energy of the lone pair (lone electron) donating to the positive metal site. The analyte lone pair energies can be evaluated from their ionization potentials or proton affinities (gas phase basicity). Co(II) and Fe(II) are paramagnetic transition metal sites which fall in the region close to Ni(II) in FIG. 15. These ions respond to a magnetic field in contrast to the Ti(IV) sites present in TiO₂ (FIG. 5). The cobalt and iron ion sites represent borderline acid sites and both respond similarly to NO. The IHSAB principle would predict that the presence of these sites would enhance the response of a sensor system.

### CONCLUSION

We have demonstrated the efficacy of a relatively small magnetic field to enhance the performance of a conductometric sensor interface. The effect, which is manifest for those transition metals that possess magnetic moments, may have potential significant implications for the development of small-scale sensor devices employing paramagnetic ion distributions to enhance sensor response in small magnetic fields. Future experiments will more closely analyze the effect of magnetic field strength on sensor response.

### REFERENCES

¹J. L. Gole and S. Ozdemir, Chem. Phys. Chem. 11, 2573 (2010).
²J. Campbell, J. A. Como, N. Larsen, and J. L. Gole, Journal of Electrochemical Society 155, D128 (2008).
3J. Gole, S. Ozdemir, and T. S. Osburn ECS Transactions 33(8), 239 (2010); S. Ozdemir and J. Gole, ECS Transactions 33(8), 111 (2010); J. L. Gole, S. Ozdemir, S. M. Prokes, and D. A. Dixon, MRS Proceedings 1257-009-04 (2010).
⁴S. Ozdemir, T. Osburn, and J. L. Gole, Journal of the Electrochemical Society 158(7), J201 (2011).
⁵J. L. Gole and W. I. Laminack, “General Approach to Design and Modeling of Nanostructure Modified Semiconductor and Nanowire Interfaces for Sensor and Microreactor Applications, Chemical Sensors: Simulation and Modeling, Volume 3—Solid State Sensors. G. Korotcenkov, Ed. (Momentum Press, New York, 2012) p. 85-136.
⁶R. G. Pearson, Coordin. Chem. Rev. 100, 403 (1990); J. Am. Chem. Soc. 85, 3533 (1963); Chemical Hardness (John Wiley VCH, Weinheim, 1997); J. Chem. Sci. 117(5), 369 (2005); Inorg. Chem. 27, 734 (1988).
⁷R. G Parr and R. G Pearson, J. Am. Chem. Soc. 105, 7512 (1983); R. G Parr and W. Yang. Density Functional Theory of Atoms and Molecules (Oxford University Press, New York, 1989).
⁸R. G. Parr, R. A. Donnelly, M. Levy and W. E. Palke, J. Chem. Phys. 68, 3801 (1978); W. Yang and R. G. Parr, Proc. Natl. Acad. Sci. USA 82, 6723 (1985); W. Yang; R.G. Parr and R. Pucci, J. Chem. Phys. 81, 2862 (1984); R. G. Parr and W. J. Yang, J. Am. Chem. Soc. 106, 4049 (1984).
⁹M. H. Cohen and A. Wasserman, J. Phys. Chem. 111, 2229 (2007).
¹⁰M. Verdaquer and F. Villain, How Molecules Become Magnetic . . . and the Resulting Wonderland, Laboratoire Chimie Inorganique et Matériaux Moléculaires, UniversitéPierre et Marie Curie, Paris VI, France, Note especially page 18 and Figure 29.
¹¹J. L. Gole, E. C. Goude, and W. Laminack, ChemPhysChem, 13, 549 (2012).
¹²S. Ozdemir and J. L. Gole, Sensors and Actuators B 151, 274 (2010).
¹³5. Lust and C. Levy-Clement, Phys. Stat. sol. (a) 182, 17 (2000).
¹⁴C. Levy-Clement, A. Lagoubi, and M. Tomkiewicz, J. Electrochem. Soc. 141, 958 (1994).
¹⁵H. Foell, M. Christophersen, J. Carstenson, and G. Hasse, Materials Science and Engineering R280, 1 (2002).
¹⁶W. I. Laminack and J. L. Gole, Nanomaterials 3, 469-485; doi:10.3390/nano3030469 (2013).
¹⁷J. L. Gole, J. D. Stout, C. Burda, Y. B. Lou, and X. B. Chen, J. Phys. Chem. B. 108, 1230 (2004); X. B. Chen, Y. B. Lou, A.C.S. Samia, C. Burda, and J. L. Gole, Adv. Funct. Mater. 15, 41 (2005).
¹⁸J. L. Gole, S. M. Prokes, and O. J. Glembocki, J. Phys. Chem. C 112(6), 1782 (2008); J. L. Gole, S. M. Prokes, X. Qiu, C. Burda, and O. J. Glembocki, Nanoscale 2, 1134 (2010).
¹⁹D. Gatteschi, R. Sessoli, J. Villain, Molecular Nanomagnets (Oxford University Press, Oxford, 2006); J. S. Miller, M. Drillon, MagnetoScience: Molecules to Materials, (Wiley-VHC, Weinheim, 2000) p. 2001-2005; M. Verdaguer and M. Matsushita, Polyhedron, 24, 2900 (2005); (d) T. Mallah et al., Science 262, 1554 (1993); R. Garde, F. Villain, and M. Verdaguer, J. Am. Chem. Soc. 124, 10531 (2002); R. Lescouëzec, et al., Angewandte Chem. 42, 1483 (2003); G. Molnár et al, Adv. Materials 19, 2163-2167 (2007); N. Roch et al., Nature 453, 633 (2008).
²⁰M. N. Rudden and J. Wilson, Elements of Solid State Physics, (John Wiley and Sons, Chichester, 1993).
²¹L. J. van der Pauw, Philips Research Reports 13, 1 (1958).
²²L. J. van der Pauw, Philips Technical Review 20: 220 (1958).
²³W. I. Laminack and J. L. Gole, “Direct in-situ Nitridation of Nanostructured Metal Oxide Deposited Semiconductor Interfaces: Decreasing the Lewis Acidity of Reversibly Interacting Sensor Sites,” submitted.
²⁴W. Laminack and J. Gole, “The IHSAB Principle and Sulphur-CHₓ Functionalized Metal Oxide Nanostructures: Reversible Chemical Sensing of NH₃”, submitted.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding based on numerical value and the measurement techniques. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A device, comprising:
a magnetic system including a magnet, and
a conductometric porous silicon gas sensor positioned relative to the magnet so that the sensor is exposed to a magnetic field of the magnet, wherein the conductometric porous silicon gas sensor includes a silicon substrate having a porous silicon layer, wherein a plurality of magnetic nanostructures are disposed on a portion of the porous silicon layer to provide a fractional coverage on the porous silicon layer, wherein the conductometric porous silicon gas sensor is operative to transduce the presence of a gas into an impedance change when exposed the magnetic field, wherein the impedance change correlates to the gas concentration.

2. The device of claim 1, wherein the magnetic nanostructure is a paramagnetic, ferrimagnetic, or ferromagnetic nanostructure.

3. The device of claim 1, wherein the magnetic nanostructure has a paramagnetic property, ferromagnetic property, or ferromagnetic property.

4. The device of claim 1, wherein the magnetic nanostructure includes a metal selected from the group consisting of: tin, iron, nickel, titanium, cobalt, platinum, palladium, osmium, rhodium, ruthenium, molybdenum, aluminum, iridium, barium, calcium, cerium, dysprosium, erbium, europium, galodium, holmium, lithium, magnesium, manganese, molybdenum, samarium, sodium, strontium, termium, thulium, tungsten, and zircomium, where the specific magnetic nanostructure including the metal has a magnetic property.

5. The device of claim 4, wherein the magnetic property is selecte from the group consisting of: a paramagnetic property, ferromagnetic property, and ferromagnetic property.

6. The device of claim 1, wherein the magnetic field at the porous silicon layer is about 200 to 1000 Gauss.

7. The device of claim 1, wherein the silicon substrate is an n-type silicon substrate.

8. The device of claim 1, wherein the silicon substrate is a p-type silicon substrate.

9. The device of claim 1, wherein the nanostructure is a nanoparticle.

10. A method of detecting a concentration of a gas, comprising:
providing a magnetic system including a magnet and a conductometric porous silicon gas sensor positioned relative to the magnet so that the sensor is exposed to a magnetic field of the magnet, wherein the conductometric porous silicon gas sensor including a silicon substrate having a porous silicon layer, wherein a plurality of magnetic nanostructures are disposed on a portion of the porous silicon layer to provide a fractional coverage on the porous silicon layer, wherein the conductometric porous silicon gas sensor is operative to transduce the presence of a gas into an impedance change when exposed to the magnetic field, wherein the impedance change correlates to the gas concentration:
exposing the porous silicon layer to the magnetic field so that the porous silicon layer is about 200 to 1000 Gauss;
introducing the gas to the sensor; and
measuring an impedance change in the sensor.

11. The method of claim 10, wherein the magnetic nanostructure is a paramagnetic, ferrimagnetic, or ferromagnetic nanostructure.

12. The method of claim 10, wherein the magnetic nanostructure has a paramagnetic property, ferromagnetic property, or ferromagnetic property.

13. The method of claim 10, wherein the magnetic nanostructure is selected from the group consisting of: tin, iron, nickel, titanium, cobalt, platinum, palladium, osmium, rhodium, ruthenium, molybdenum, aluminum, iridium, barium, calcium, cerium, dysprosium, erbium, europium, galodium, holmium, lithium, magnesium, manganese, molybdenum, samarium, sodium, strontium, termium, thulium, tungsten, and zircomium, where the specific magnetic nanostructure including the metal has a magnetic property.

14. The method of claim 10, wherein the magnetic property is selected from the group consisting of: a paramagnetic property, ferromagnetic property, and ferromagnetic property.

15. The method of claim 10, further comprising: correlating the impedance change to the concentration of the gas, wherein correlating includes computing a magnitude of the impedance change, computing the time over which the magnitude of the impedance change occurs, and computing a slope from the ratio of the magnitude of the impedance change and the time to determine the concentration of the gas.

15. The method of claim 10, wherein the silicon substrate is an n-type silicon substrate.

16. The method of claim 10, wherein the silicon substrate is a p-type silicon substrate.
